(19) United States
(12) Plant Patent Application Publication
(10) Pub. No.: US 2026/0033410 P1
(43) Pub. Date: Jan. 29, 2026

(54) Cherry Tree Named 'V69068'

(50) ;

(71) Applicant: University of Guelph, Guelph (CA)

(72) Inventors: Ghassem Tehrani, Guelph (CA); James William Lay, Guelph (CA)

(21) Appl. No.: 19/327,359

(22) Filed: Sep. 12, 2025

Related U.S. Application Data
(63) Continuation-in-part of application No. 18/783,946, filed on Jul. 25, 2024.

(51) Int. Cl. A01H 6/74; A01H 5/00; A01H 5/08

(52) U.S. Cl. CPC A01H 6/7445; A01H 5/00; A01H 5/08
USPC PLT/181

(57) ABSTRACT

A new cherry tree particularly distinguished by red, flat, round fruit, with a highly precocious fruiting habit, self-fertility, less susceptible to rain crack and moderate resistance to bacterial canker, is disclosed.

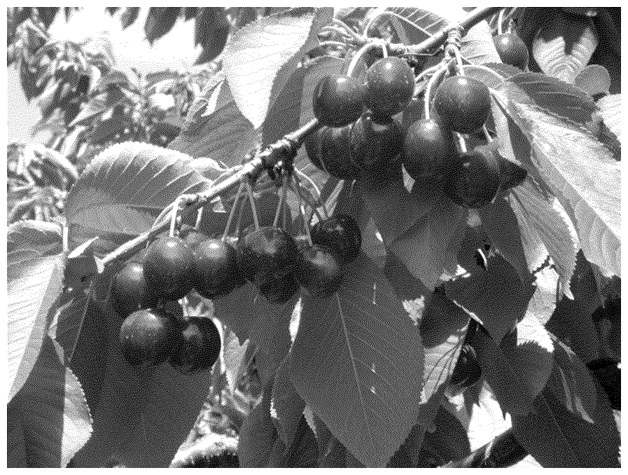

### CROSS REFERENCE TO RELATED APPLICATION

The present application is a continuation in part of and claims priority to U.S. application Ser. No. 18/783,946, as filed on Jul. 25, 2024, the entire contents of which are incorporated herein by reference for all purposes.

Genus and species: Prunus avium.

Variety denomination: ‘V69068’.

### BACKGROUND OF THE NEW PLANT

The present invention comprises a new and distinct variety of cherry tree, botanically known as Prunus avium and hereinafter referred to by the variety name ‘V69068’. ‘V69068’ originated from a controlled cross conducted in May 1969 between the female parent variety ‘Van’ (unpatented) and the male parent ‘Stella’ (unpatented). ‘V69068’ was bred and developed in Vineland, Ontario, Canada. The resulting seeds were germinated and one resulting seedling named ‘V69068’ was selected. Cherry cultivar ‘V69068’ was first asexually reproduced in Vineland, Onterio. Cherry cultivar ‘V69068’ was found to reproduce true to type in successive generations of asexual propagation via bud wood cuttings.

### SUMMARY

The following are the most outstanding and distinguishing characteristics of this new variety when grown under normal horticultural practices in Wenatchee, Washington.
1. Red, flat, round fruit;
2. Highly precocious fruiting habit;
3. Self-fertile variety;
4. Less susceptible to rain induced cracking; and
5. Moderate resistance to bacterial canker.

### DESCRIPTION OF THE PHOTOGRAPH

This new cherry tree is illustrated by the accompanying photograph showing the fruit and leaves of ‘V69068’ from June 2019 on five-year-old plants grown in Wenatchee, Washington. The colors shown are as true as can be reasonably obtained by conventional photographic procedures.

### DESCRIPTION OF THE NEW VARIETY

The following detailed descriptions set forth the distinctive characteristics of ‘V69068’. The data which define these characteristics was collected in Wenatchee, Washington on five-year-old trees. All colour determinations were made using 2015 Royal Horticultural Society (RHS) Colour Chart, 6^{th} edition.
Classification:
   Family.—Rosaceae.
   Species.—Prunus avium.
   Denomination.—‘V69068’.
Parents:
   Female.—‘Van’ (unpatented).
   Male.—‘Stella’ (unpatented).
General:
   Height and spread.—3.0 m×2.0 m.
   Cold hardiness.—Zone 5.
   Diseases and pests.—Less susceptible to rain-induced cracking. Moderately resistant to bacterial canker.
   Rootstock.—Planted out on Gisela 6 (U.S. Plant Pat. No. 8,954).
   Propagation.—Bud wood in August.
   Growth rate.—1.0 m per season.
   Plant habit.—Spreading.
   Branching.—Medium.
   Habit.—Spreading.
   Fruiting habit.—Highly precocious.
Trunk description:
   Size of trunk.—7.5 centimeters at 30 centimeter height.
   Bark color.—Young stems: Color: RHS 144B Strong Yellow Green. Older bark: Color: RHS N200A Dark Grayish Yellow Brown.
   Lenticels.—Yellowish Gray RHS 156A.
   Description of dormant shoots (one year-old).—Pubescence: None. Shoot size: Length: 1.0 cm. Width: 0.5 cm. Anthocyanin presence: None. Thickness of shoot at center of middle internode: 1.0 cm. Shoot angle: 45 Degrees. Branch internode length: 15.0 cm.
   Vegetative buds.—Length: 1.0 cm. Width: 0.5 cm. Shape: Oval.
   Color of growing tip of shoot.—RHS 144B Strong Yellow Green.
   Pubescence of shoots tip.—Absent.
   Anthocyanin presence.—Absent.
Foliage description:
   Leaf shape.—Elliptical.
   Leaf division.—Simple.
   Leaf base.—Obtuse with two glands at the base.
   Leaf apex.—Acute.
   Leaf fragrance.—Absent.
   Leaf venation.—Type: Dichotomous. Color: RHS 146A Upper Surface, RHS 146B Lower Surface.
   Leaf margins.—Serrate.
   Leaf arrangement.—Alternate.
   Leaf aspect.—0.5° Leaf attachment: Petiolate.
   Leaf surface.—Smooth.
   Leaf size.—Length: 13.5 cm. Width: 6.5 cm.
   Leaf color.—Newly expanded leaves: Upper surface: RHS 146B. Lower surface: RHS 146A. Mature leaves: Upper surface: RHS 146A. Lower surface: RHS 146B.
Petioles:
   Length.—3.5 cm.
   Width.—0.2 cm.
   Color.—RHS 146C.
   Anthocyanin.—Absent.
   Texture.—Smooth.
Inflorescence description:
   Blooming period.—April 5 to 12 in Osoyoos, British Columbia, Mid-season.
   Lastingness of inflorescence.—7 days.
   Flower quantity.—Moderate.
   Flower fragrance.—Cherry like fragrance.
   Flower type.—Raceme.
   Flowers per raceme.—One.
   Flower size (diameter).—1.5 cm.
Sepals:
   Number.—Five.
   Shape.—Conical.
   Base.—Pointed.
   Apex.—Pointed.
   Margin.—Entire.
   Color.—Upper surface: RHS 139C. Lower surface: RHS 138C.
   Texture.—Upper surface: Smooth. Lower surfaces: Smooth.
   Length.—0.6 cm.
   Calyx width.—0.5 cm.
Petals:
   Number.—Five.
   Arrangement.—Intermediate.
   Shape.—Obovate.
   Base.—Obtuse.
   Apex.—Acute.
   Margin.—Entire.
   Length.—1.7 cm.
   Width.—0.6 cm.
   Color.—RHS N155B.
Pedicels:
   Length.—3.0 cm.
   Width.—0.2 cm.
   Color.—RHS 143B.
   Texture.—Smooth.
Rachis: Absent.
Reproductive organs:
   Gynoecium.—Pistil: Number: One. Length: 0.6 cm. Style: Color: RHS 143C. Length: 0.6 cm. Width: 0.1 cm. Stigma: Diameter: 0.1 cm. Color: RHS 142A. Ovary: Color: RHS 143C.
   Androecium.—Filaments: Color: RHS 155D. Length: 1.0 cm. Anthers: Color: RHS 15C. Pollen: Amount: Large amount. Color: RHS 15C.
Fertility: Self Fertile.
Fruit description:
   Fruit type.—Drupe.
   Fruit shape.—Flat Round.
   Fruit apex.—Flat.
   Fruit base.—Medium cordate.
   Fruit size.—Length: 3.0 cm. Width: 2.8 cm.
   Fruit firmness.—Firm; 435 g/mm.
   Fruit set.—Good.
   Fruit stalk.—Length: 4.0 cm. Thickness: 0.2 cm. Surface: Smooth. Color: RHS 143B.
   Fruit bearing.—Yes.
   Number of fruit per cluster.—Three.
   Lenticels.—Very small to absent.
   Overall skin color.—RHS N186A.
   Ripening time.—Three days after ‘Bing’ variety.
   Harvest date.—June 30 Osoyoos, British Colombia, three days after ‘Bing’.
   Skin surface.—Smooth.
   Skin thickness.—Medium.
   Flesh color.—RHS N186A (no secondary flesh color).
   Juice.—Description: Color: RHS N186A.
   Flesh texture.—Medium.
   Apex end.—Color: RHS N186A.
   Fruit use.—Fresh market.
   Weight.—13 grams.
   Brix.—18.
   Acidity.—0.78 g/100 g.
   Firmness.—435 g/mm.
   Storage life.—6 weeks.
   Productivity.—20,000 lbs. per acre.
Seeds:
   Number per berry.—One.
   Color.—RHS N165B.
   Size.—Length: 1.0 cm. Width: 0.8 cm.
   Shape.—Elliptical.

### COMPARISON WITH PARENTAL LINES AND KNOWN VARIETY

‘V69068’ is distinguished from its parents as described in Table 1.

TABLE 1
Comparison with Parents

| Characteristic | ‘V69068’ | Female parent | Male parent |
|---|---|---|---|
| Self-Fertility | Yes | No | Yes |
| Rain Crack Resistant | Yes | No | No |

‘V69068’ is most similar to the commercial cherry named ‘Bing’ (unpatented). Differences between the two varieties are described in Table 2:

TABLE 2

| Characteristics | ‘V69068’ | ‘Bing’ |
|---|---|---|
| Self-Fertility | Yes | No |
| Bacterial Canker Resistance | Yes | No |

Table 3 below provides a comparison of ‘V69068’ with four cherry varieties, ‘V690630’, ‘V69061’, ‘V69062’, ‘V690628’, with pending US patent applications:

TABLE 3

| Cultivar | ‘V690630’ | ‘V69061’ | ‘V69062’ | ‘V690628’ | ‘V69068’ |
|---|---|---|---|---|---|
| Ripening | 10 days | 10 days | Same as | Three days | Three days |
| Time | before the | before the | variety | before | after Bing |
| | ‘Bing’ | variety | ‘Bing’ | variety | varieties |
| | variety | ‘Rainier’ | | ‘Bing’ | |
| Fruit Size | 29 | 29 | 30 | 30 | 27 |
| mm | | | | | |
| Fruit Skin | N186A | N45 | N186A | N186A | N186A |
| Colour | | | | | |
| Firmness | 711 | 417 | 520 | 413 | 435 |
| g/mm | | | | | |
| Brix | 18 | 17 | 18 | 18 | 18 |
| Acidity | 0.80 | 0.68 | 0.78 | 0.55 | 0.78 |
| g/100 g | | | | | |
| Fruit Shape | Kidney to | Kidney | Flat-round | Kidney to | Flat-round |
| | flat-round | | | flat-round | |
| Growth | Spreading | Spreading | Spreading | Spreading | Spreading |
| Habit | | | | | |
| Fruiting | Precocious | Precocious | Very | Precocious | Highly |
| Habit | | | Precocious | | Precocious |
| Stem Length | Short | Medium- | Medium | Medium | Long |
| | | Long | | | |
| Flesh colour | Black-red | Yellow | Dark red | Dark red- | Red |
| | | | | purple | |
| Disease | Moderately | Moderately | Moderately | Moderately | Moderately |
| | resistant to | resistant to | resistant to | resistant to | resistant to |
| | bacterial | bacterial | bacterial | bacterial | bacterial |
| | canker | canker | canker | canker | canker |
| Rain- | Less | Extremely | Very | Good | Less |
| induced | susceptible | Tolerant | Tolerant | resistance | susceptible |
| cracking | | | | | |

I claim:

1. A new and distinct variety of cherry tree designated ‘V69068’ as illustrated and described herein.
